(19) United States
(12) Patent Application Publication
(10) Pub. No.: US 2022/0406438 A1
(43) Pub. Date: Dec. 22, 2022

(54) SYSTEMS AND METHODS FOR NUTRIENT SCORING ACCORDING TO LABELLED NUTRIENTS ON A FOOD OR BEVERAGE PRODUCT

(71) Applicant: SOCIETE DES PRODUITS NESTLE S.A., Vevey (CH)

(72) Inventors: MATHILDE GRESSIER, Linars (FR); GABRIEL MASSET, Yverdon-les-Bains (CH); TSZ NING MAK, Le Mont-Sur-Lausanne (CH)

(21) Appl. No.: 17/755,110

(22) PCT Filed: Oct. 26, 2020
(86) PCT No.: PCT/EP2020/080020

§ 371 Date: Apr. 21, 2022

(30) Foreign Application Priority Data
Oct. 29, 2019 (EP) 19206003.6

(51) Int. Cl. G16H 20/60

(52) U.S. Cl. CPC G16H 20/60

(57) ABSTRACT

The present invention relates to systems and methods for nutrient scoring according to labelled nutrients on a food or beverage product. The advantage of the present invention and its systems and methods for nutrient scoring is that it permits a simplified way of assessing the nutrient profile of a food or beverage product only based on the labelled nutrients.

### FIELD OF THE INVENTION

The present invention relates to systems and methods for nutrient scoring according to labelled nutrients on a food or beverage product. One advantage of the present invention and its systems and methods for nutrient scoring is that it permits a simplified way of assessing the nutrient profile of a food or beverage product only based on the labelled nutrients. Another advantage is that it allows to the user to compare, for example, similar food or beverage products based only on the labelled nutrients to determine a single nutrient score for each product.

### BACKGROUND TO THE INVENTION

Nutrient profiling (NP) models exist to classify foods and food products according to their nutritional quality. Commonly, such models include assessing a number of negative nutrients, positive nutrients and total ingredients in a product. Ingredients and nutrients information are not always available on food labels on food products, therefore an estimation of nutrient and ingredient content based on similar food products is often needed. Given that nutrient and ingredient information is not always available, assessing the nutritional quality of products using complex nutrient profiling systems with incomplete nutrient and/or ingredient information can have undesirable effects.

There is a need to have a simple and quick method to assess food products based on only labelled nutrients. The application of such quick assessment can have vast implications from helping consumers choose healthier products to reformulation of products within the food and beverage industry.

There are methods for calculating nutritional quality based on food labels but they often omit the importance of dietary fibre. Dietary fibre is found mainly in fruits, vegetables, whole grains and legumes and it has health benefits of helping to maintain a healthy weight and lowering your risk of diabetes, heart disease and some types of cancer.

There is a need to simplify the assessment of nutritional quality of products so that the consumers can more easily identify the nutritional quality of food and beverage products. In particular, there is a need to easily identify healthy food and beverage products based on labelled nutrients on a product.

The present invention provides a novel solution to enable more accurate and transparent assessment of food products by providing a single product nutrient score which requires only the commonly labelled nutrient information.

### SUMMARY OF THE INVENTION

The present disclosure relates to novel systems and methods for nutrient scoring according to labelled nutrients on a food or beverage product.

In several embodiments, the systems and methods allow for a single nutrient score per product which allows for easy comparison between different products, for example, between different food products or between different beverage products.

The novel addition of a fibre component in the score improves the accuracy of the assessment of the nutrient profile.

The invention further relates to a computer implemented system and computer program product for carrying out the inventive method.

The features and advantages described herein are not all exclusive, and many additional features and advantages will be apparent to one ordinarily skilled in the art in view of the figures and the description.

### DESCRIPTION OF FIGURES

FIG. 1 represents one embodiment of a system for calculation of a single nutrient product nutrient score for food and beverage products.

Technical features of the system include:
Product label input module (100)
Fibre module (101)
Protein module (102)
Fat module (103)
Sugar module (104)
Sodium module (105)
Single Product Nutrient Score module (106)
Comparison module (107)
Recommendation module (108)
User interface display module (109)

Detailed descriptions of the modules of the system are described below.

### DETAILED DESCRIPTION OF THE INVENTION

### Definitions

### Nutrient

In some embodiments, the term “nutrient” as used herein refers to compounds having a beneficial effect on the body e.g. to provide energy, growth or health. The term includes organic and inorganic compounds. As used herein the term nutrient may include, for example, macronutrients, micronutrients, essential nutrients, conditionally essential nutrients and phytonutrients. These terms are not necessarily mutually exclusive. For example, certain nutrients may be defined as either a macronutrient or a micronutrient depending on the particular classification system or list.

In various embodiments, the term “macronutrient” is used herein consistent with its well understood usage in the art, which generally encompasses nutrients required in large amounts for the normal growth and development of an organism. Macronutrients in these embodiments may include, but are not limited to, carbohydrates, fats, proteins, amino acids and water. Certain minerals may also be classified as macronutrients, such as sodium, chloride, or calcium.

In various embodiments, the term “micronutrient” is used herein consistent with its well understood usage in the art, which generally encompasses compounds having a beneficial effect on the body, e.g. to help provide energy, growth or health, but which are required in only minor or trace amounts. The term in such embodiments may include or encompass both organic and inorganic compounds, e.g. individual amino acids, nucleotides and fatty acids; vitamins, antioxidants, minerals, trace elements, e.g. iodine, and electrolytes, e.g. sodium, and salts thereof, including sodium chloride.

In one embodiment of the invention, the preferred nutrients to be measured are: fibre, protein, fat, sugar, or sodium. Description of these specific nutrients are described below.

### User of System and Methods

In several embodiments, the user is a typical consumer of food and beverage products.

In some embodiments, the user is a retailer who may be interested in optimizing their stock of food and beverage products in consideration of higher nutrient content.

In some embodiments, the user is a health care professional who is interested in recommending food and beverage products with high nutrient content.

In some embodiments, the user is interested in food and beverage products for human use.

In other embodiments, the user is interested in food and beverage products for animal use, particularly companion animals such as dogs and cats.

### System

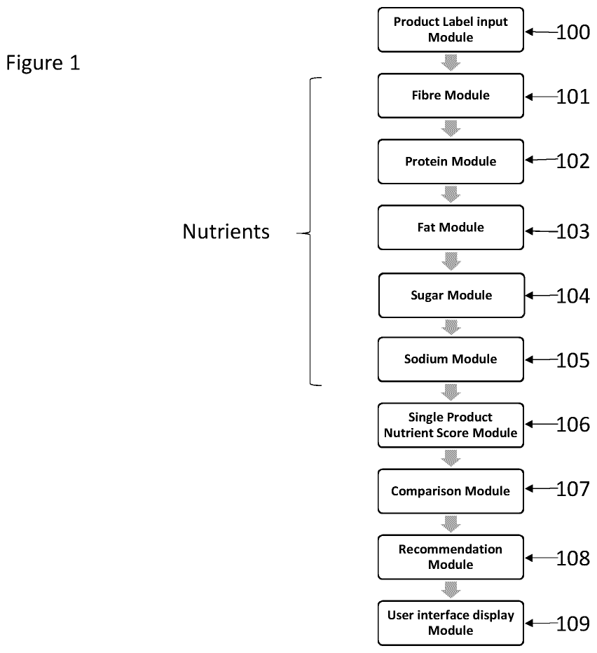

FIG. 1 provides an example of a system for calculation of a single product nutrient score for food and beverage products with various modules which are described below.

### Product Label Input Module

In some embodiments, one or more devices carried by the consumer could provide real-time information to the system when the consumer is in a food purchasing establishment such as a grocery store or a restaurant. Devices such as RFID readers, NFC readers, wearable camera devices, and mobile phones could receive or determine (such as by scanning RFID tags, reading bar codes, or determining the physical location of a user) foods that are available to a user at a particular grocery store or restaurant.

FIG. 1 provides an example of a Product Input Module (100).

In one embodiment, the system provides recommendations in real-time taking into account what foods or food products could be immediately purchased or consumed by the user.

In some embodiments, when a consumer enters a grocery store the disclosed system may push information to the user's mobile phone recommending that the consumer select certain items from the available food products to optimize the user's nutrient score for a given time period.

In still other embodiments, a voice recognition feature recognizes inputs provided vocally by a user. In some embodiments, the voice recognition system enables the user to speak directly the items he or she has consumed or will consume.

In another embodiment, the disclosed system could use geolocation to provide appropriate recommendations based on the user's location. For example, an app on a user's phone, tablet, or computer could provide the user (e.g., in a chat box) different activity tips if the user is at work, in a gym, or at home.

In some embodiments, the disclosed system includes or is connected to a database containing food and beverage products and their respective nutrient content. In this embodiment, the disclosed system includes a fuzzy search feature that enables a user to enter a consumed (or to-be consumed) food or beverage product, and thereafter searches the database to find a closest item to the user-provided item. The disclosed system, in this embodiment, uses stored nutritional information about the matched food item to determine a product nutrient score as described in the present invention.

In various embodiments, the disclosed system further includes an interface (e.g., a graphical user interface) to display the amount of each nutrient available in each food or beverage product. In some embodiments, this interface enables users to choose the foods or beverages to be consumed, and correspondingly displays a nutrient score based on the modified food or beverage to be consumed. In other embodiments, the system is configured to the food or beverage to be consumed using data, such as by scanning one or more bar codes, QR codes, or RFID tags, or by tracking items ordered from a menu or purchased at a grocery store.

In some embodiments, the disclosed system includes a recommendation feature that recommends particular foods or beverages to a user based on their nutrient score. In a preferred embodiment, the system recommends foods or beverages with the best nutrient score.

In various embodiments, the disclosed system stores some or all of the values needed to calculate nutrient scores in one or more databases.

### Product Nutrient Score Calculation

In various embodiments, the fibre, protein, fat, sugar and sodium measurement parameters for calculating the single product nutrient score are chosen from those available on a food or beverage product label.

In some embodiments, the fibre, protein, fat, sugar and sodium measurement parameters for calculating the product nutrient score may be estimated based on similar product information.

For example, the system and methods of the present invention can be used for comparing two or more food or beverage products and choosing the food or beverage product with the highest product nutrient score.

In a preferred embodiment, the nutrients to be calculated are selected from the group consisting of: fibre, protein, fat, sugar and sodium.

### Fibre and Fibre Module

Fibre is dietary material containing substances such as cellulose, lignin, and pectin, that are resistant to the action of digestive enzymes. There are two main types of fibre: soluble and insoluble fibre.

Soluble fibre dissolves in water to form a gel-like material. It can help lower blood cholesterol and glucose levels. Soluble fibre is found in oats, peas, beans, apples, citrus fruits, carrots, barley and psyllium.

Insoluble fibre promotes the movement of material through your digestive system and increases stool bulk, so it can be of benefit to those who struggle with constipation or irregular stools. Whole-wheat flour, wheat bran, nuts, beans and vegetables, such as cauliflower, green beans and potatoes, are good sources of insoluble fibre.

In a preferred embodiment, fibre is calculated per 100 kcal units.

FIG. 1 provides an example of the Fibre Module (101).

### Protein and Protein Module

Proteins are nitrogenous organic compounds composed of one or more long chains of amino acids. There are two categories of amino acids: essential amino acids and non-essential amino acids.

Essential amino acids are those that cannot be made by the body and must be obtained from food.

Protein is found in foods from both animal and plant sources. Beans, peas, nuts, seeds, soy products, dairy products, eggs, seafood, meat and poultry are good sources of protein.

In a preferred embodiment, protein is calculated per 100 kcal units.

FIG. 1 provides an example of the Protein Module (102).

### Fat and Fat Module

Fats, a subgroup of lipids, are also known as triglycerides, meaning their molecules are made from one molecule of glycerol and three fatty acids. Fats provides calories and helps the body absorb certain vitamins, cushions and insulates the body, and supports many body processes.

Examples of fats are: saturated fats are usually solid at room temperature. They are usually found in animal fats, meat, animal and full-fat dairy products. Certain tropical plant oils, such as coconut oil, palm oil, and palm kernel oil are high in saturated fat.

Monounsaturated and polyunsaturated fats are found in higher proportions in plants and seafood and are usually liquid at room temperature. Monounsaturated fats can be found in avocados, nuts, seeds, vegetable oils and margarine; polyunsaturated fats can also be found in such foods as well as fatty fish such as salmon, herring and mackerel.

Trans-fat is naturally found in small amounts in some animal products such as meat, whole milk, and milk products, however, it is also produced during a process called hydrogenation from vegetable oils. Trans-fat is linked to increased risk of coronary heart disease and early mortality. Trans-fat can be found in cakes, cookies, crackers, icings, margarines.

In a preferred embodiment, fat is calculated per 100 g units.

FIG. 1 provides an example of the Fat Module (103).

In the equations, fat is signified by SFA.

### Sugar and Sugar Module

Sugars are soluble, crystalline, typically sweet-tasting carbohydrates. Examples of sugars are: fructose, galactose, glucose, lactose, maltose, and sucrose. Sugars can be naturally occurring in foods such as fruit (fructose) and milk (lactose), or added to foods and beverages for taste, texture and preservation. Sugars are often found in foods such as cake and desserts, sugar-sweetened beverages, and sweets.

In a preferred embodiment, sugar is calculated per 100 g units. Sugars are calculated as total sugars (T.sugar).

FIG. 1 provides an example of the Sugar Module (105).

### Sodium and Sodium Module

Sodium is a mineral and one of the chemical elements found in salt. Salt is also known by its chemical name, sodium chloride. Sodium can increase the risk of developing high blood pressure and cardiovascular disease.

Sodium is usually added to food during processing. Top sources of sodium are breads, pizza, cold cuts and cured meats, savory snacks and cheese.

In a preferred embodiment, sodium is calculated per 100 g units.

FIG. 1 provides an example of the Sodium Module (105).

### Single Product Nutrient Score Module

In several embodiments, the single product nutrient score is a single score calculated from a plurality of nutrients of measured from the product label.

In one embodiment, the nutrients used to calculate single product nutrient score are selected from the group consisting of: fibre, protein, fat, sugar, and sodium.

In a preferred embodiment, the nutrients used to calculate the single nutrient score are consisting of: fibre, protein, fat, sugar, and sodium.

In several embodiments, the protein and fibre nutrients are each calculated per 100 kcal units.

In several embodiments, the fat, sugar and sodium are each calculated according to 100 g units.

In a preferred embodiment, the single product nutrient score is calculated from:
a fibre calculation module configured to calculate the amount of fibre for a product per 100 kcal;
a protein calculation module configured to calculate the amount of protein for a product per 100 kcal;
a fat calculation module configured to calculate the amount of fat for a product per 100 g;
a sugar calculation module configured to calculate the amount of sugar for a product per 100 g;
a sodium calculation module configured to calculate the amount of sodium for a product per 100 g;

to result in a single product nutrient score based on the input of nutrient scores from the modules for fibre, protein, fat, sugar, sodium.

In another embodiment, the single product nutrient score is connected to a user interface display module to display product nutrient scores for one product or a plurality of products.

In a preferred embodiment, the product nutrient score is calculated according to the formula:

$$\frac{( \frac{{PROTEIN}_{100 kcal}}{50} + \frac{2 * {FIBRE}_{100 kcal}}{25} )}{2} - \frac{( \frac{{SFA}_{100 g}}{20} + \frac{T . {SUGARS}_{100 g}}{90} + \frac{{SODIUM}_{100 g}}{2000} )}{3}$$

In another preferred embodiment, the product nutrient score is calculated according to the formula:

$$( \frac{{PROTEIN}_{100 k c a l}}{50} ) - \frac{( \frac{{SFA}_{100 g}}{20} + \frac{T . {SUGARS}_{100 g}}{90} + \frac{{SODIUM}_{100 g}}{2000} )}{3}$$

In yet another embodiment, methods for selecting a food or beverage product with the highest product nutrient are provided by the invention.

In a preferred embodiment, two or more food or beverage products may be compared by their product nutrient score and a recommendation of the food or beverage product with the highest nutrient score may be given to the user.

In several embodiments, the recommendation of the food or beverage product with the highest nutrient score may be combined with other data on user preferences or product costs.

FIG. 1 provides an example of the Single Product Nutrient Score Module (106).

### Comparison Module

In several embodiments, the product nutrient score may be used for comparing a plurality of products against one another.

In one embodiment, food products of the same category may be compared against each other.

In another embodiment, beverage products of the same category may be compared against each other.

For example, food or beverage products may be compared within a category such as those categories described by the Codex Alimentarius Commission (2019):

01.0 Dairy products and analogues, excluding products of category 02.0

02.0 Fats and oils, and fat emulsions

03.0 Edible ices, including sherbet and sorbet

04.0 Fruits and vegetables (including mushrooms and fungi, roots and tubers, pulses and legumes, and aloe vera), seaweeds, and nuts and seeds

05.0 Confectionery

06.0 Cereals and cereal products, derived from cereal grains, from roots and tubers, pulses, legumes and pith or soft core of palm tree, excluding bakery wares of food category 07.0

07.0 Bakery wares

08.0 Meat and meat products, including poultry and game

09.0 Fish and fish products, including mollusks, crustaceans, and echinoderms

10.0 Eggs and egg products

11.0 Sweeteners, including honey

12.0 Salts, spices, soups, sauces, salads, protein products

13.0 Foodstuffs intended for particular nutritional uses

14.0 Beverages, excluding dairy products

15.0 Ready-to-eat savouries

16.0 Prepared foods

FIG. 1 provides an example of the Comparison Module (107).

### Recommendation Module

In several embodiments, the product nutrient score may be used for generating user recommendations in the recommendation module.

In a preferred embodiment, the product nutrient score may be used for identifying the product with the highest nutrient score in the recommendation module.

In other embodiments, the product nutrient score may be combined with comparisons of product price per volume, per mass, per package to recommend the product which has the highest product nutrient score combined with the lowest cost.

In other embodiments, the product nutrient score may be combined with availability of the product in the store for product recommendation in the recommendation module.

In other embodiments, the product nutrient score may be combined with the highest feedback score from previous consumers who like the product.

In other embodiments, the product nutrient score may be combined with the user history to determine whether the user has selected the product in the past and suggest a new product.

In other embodiments, the product nutrient score may be combined with the user history to determine whether the user has selected the product in the past and suggest the same product.

In several embodiments, the recommendation module communicates to the user the product with the highest score.

FIG. 1 provides an example of the Recommendation Module (108).

### User Interface Display Module

The user interface display may be a liquid crystal display (LCD), a suitable projector, or any other suitable type of display, including audio user interfaces. The user interface display may be used to display information about the different nutrient modules, for example, the modules for protein, fibre, fat, sugar, and sodium calculation for a given product as well as the single product nutrient score.

It may also contain access to a database of product nutrient scores for other food and beverage products, a database of users including user preferences of certain products, a database of previously generated user product nutrient scores, and/or databases to enable an administrator at the device to interact with the other databases described above.

FIG. 1 provides an example of the User Interface Display Module (109).

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media.

The instructions may be provided as software or firmware, and may be implemented in whole or in part in hardware components such as ASICs, FPGAs, DSPs, or any other similar devices.

The instructions may be configured to be executed by one or more processors, which when executing the series of computer instructions, performs or facilitates the performance of all or part of the disclosed methods and procedures.

### EXAMPLES

### Example 1: Product Nutrient Score Optimisation

An analysis of 18 422 products across 60 countries in the Mintel Global New Products Database (GNPD) identified the most frequently labelled nutrients on products were: protein (85.4%), carbohydrates (85.2%) and total fat (83%), energy (kcal) (77.7%), energy (kJ) (56.5%), total sugars (68.5%), saturated fat (56.6%), salt or sodium (42.2% and 36.8%), and fibre (44.7%). Based on the public health implications of these nutrients, proteins and fibres were considered as “nutrient to encourage” (herein forth positive subscore), and saturated fat, total sugars and sodium as “nutrients to limit” (negative subscore) in the baseline algorithm.

The baseline algorithm was initially based on the nutrient ratios of four nutrients. Nutrient ratio was calculated by dividing the quantity of a nutrient within the food item by its daily values (DVs). DVs used were based on the WHO, CODEX, US FDA and European recommendations and were as follow: 50 g protein, 20 g SFA (fat), 90 g total sugars, and 2000 mg sodium, all based on energy intake of 2000 kcal/day. The sum of the “nutrients to limit” ratios were divided by three to give equal weights to each of the “nutrient to limit”, as well as equal weights between the positive and negative subscores. Four factors were considered before reaching the final algorithm (Table 1):

1) The reference amount: Previous studies on reference amount and nutrient density suggested that nutrient profiling models based on 100 kcal or per serving were preferable for positive subscores, and per 100 g performed better for negative subscores. In our algorithm, we chose the reference amount 100 g for the negative subscore to penalise energy dense products. We tested the reference amounts (per 100 g, per 100 kcal and per serving) for positive subscore.

2) Division or subtraction: The formula used were tested in two scenarios: positive subscore minus negative subscore, and positive subscore divided by negative subscore.

3) Inclusion of fibre in the positive subscore: A limitation to include only protein as a positive nutrient in the algorithm was that foods low in protein but containing ingredients with health benefits such as fruit, vegetables and whole grain, would not rank highly in our system. Therefore, we tested the addition of fibre in the algorithm.

4) Addition of weight to fibre to emphasise the nutrient quality of fibre containing food.

Eighteen algorithms were tested to determine the final algorithm which had the best correlations with external NP systems and diet quality indicators.

TABLE 1
Steps taken to test different algorithms and determining the final score

| | Considerations | Examples of tested algorithms |
|---|---|---|
| 1 | Reference Amount (per 100 g, 100 kcal or per serving) for positive subscore | $( \frac{{PROTEIN}_{100 g}}{50} ) - \frac{( \frac{{SFA}_{100 g}}{20} + \frac{\text{T.S} {UGARS}_{100 g}}{90} + \frac{{SODIUM}_{100 g}}{2000} )}{3} \\ ( \frac{{PROTEIN}_{100 kcal}}{50} ) - \frac{( \frac{{SFA}_{100 g}}{20} + \frac{\text{T.S} {UGARS}_{100 g}}{90} + \frac{{SODIUM}_{100 g}}{2000} )}{3} \\ ( \frac{{PROTEIN}_{per serving}}{50} ) - \frac{( \frac{{SFA}_{100 g}}{20} + \frac{\text{T.S} {UGARS}_{100 g}}{90} + \frac{{SODIUM}_{100 g}}{2000} )}{3}$ |
| 2 | Testing the formula (subtraction or division) | $( \frac{{PROTEIN}_{100 g}}{50} ) / \frac{( \frac{{SFA}_{100 g}}{20} + \frac{\text{T.S} {UGARS}_{100 g}}{90} + \frac{{SODIUM}_{100 g}}{2000} )}{3}$ |
| | | $( \frac{{PROTEIN}_{100 kcal}}{50} ) / \frac{( \frac{{SFA}_{100 g}}{20} + \frac{\text{T.S} {UGARS}_{100 g}}{90} + \frac{{SODIUM}_{100 g}}{2000} )}{3}$ |
| | | $( \frac{{PROTEIN}_{per serving}}{50} ) / \frac{( \frac{{SFA}_{100 g}}{20} + \frac{\text{T.S} {UGARS}_{100 g}}{90} + \frac{{SODIUM}_{100 g}}{2000} )}{3}$ |
| 3 | Addition of fibre | $( \frac{{PROTEIN}_{100 kcal}}{50} ) - \frac{( \frac{{SFA}_{100 g}}{20} + \frac{\text{T.S} {UGARS}_{100 g}}{90} + \frac{{SODIUM}_{100 g}}{2000} )}{3}$ |
| | | $\frac{( \frac{{PROTEIN}_{100 kcal}}{50} + \frac{{FIBRE}_{100 kcal}}{25} )}{2} - \frac{( \frac{{SFA}_{100 g}}{20} + \frac{\text{T.S} {UGARS}_{100 g}}{90} + \frac{{SODIUM}_{100 g}}{2000} )}{3}$ |
| 4 | Adding weight to fibre to favour fruit, vegetable and whole grain contents | $\frac{( \frac{{PROTEIN}_{100 kcal}}{50} + \frac{{FIBRE}_{100 kcal}}{25} )}{2} - \frac{( \frac{{SFA}_{100 g}}{20} + \frac{\text{T.S} {UGARS}_{100 g}}{90} + \frac{{SODIUM}_{100 g}}{2000} )}{3}$ |
| | | $\frac{( \frac{{PROTEIN}_{100 kcal}}{50} + \frac{2 * {FIBRE}_{100 kcal}}{25} )}{2} - \frac{( \frac{{SFA}_{100 g}}{20} + \frac{\text{T.S} {UGARS}_{100 g}}{90} + \frac{{SODIUM}_{100 g}}{2000} )}{3}$ |

### Database Used to Calculate Scores

The algorithms were tested using foods from the USDA Food and Nutrition Database for Dietary Studies 2011-2012 (FNDDS). FNDDS contains nutrient information unbranded foods and beverages commonly consumed by the US population. In this study, food categories “baby food”, “supplements” and any products intended for medical purposes were excluded from analysis. Nutrition composition of 6960 food items were used for the testing of the algorithms.

### Validating Scoring System Against NP Models: Ofcom, SAIN-LIM and NRF9.3

The scores calculated based on our tested algorithms were validated against the French SAIN-LIM and UK Ofcom nutrient profiling (NP) models. Scores for SAIN-LIM and Ofcom were calculated for the same food items from the FNDDS.

The SAIN-LIM system was developed to assess nutrient quality of products for labelling and regulatory purposes in France (Darmon et al. Am J Clin Nutr, 2009. 89(4): p. 1227-36. Essentially, it is based on two subscores summarizing the positive (SAIN) nutrients per 100 kcal basis (proteins, fibre, ascorbic acid, calcium, and iron) and negative (LIM) nutrients (sodium, added sugars, and saturated fatty acids) per 100 g of food. The model classifies products into four nutrient profile classes 1. recommended for health; 2. neutral; 3. recommended in small quantities or occasionally; 4. consumption should be limited.

The UK Ofcom NP system is one of the most validated NP systems (UK. Department of Health 2011, https://www.gov.uk/government/publications/the-nutrient-profiling-model). It was developed to differentiate “healthier” and “less healthy” foods and drinks for the restriction of TV advertising for products high in fat, salt or sugar to children. Similar to the SAIN-LIM system, the Ofcom model calculates points for “A” nutrients (energy, saturated fats, total sugar and sodium) and “C” nutrients (fruit, vegetables and nut content, fibre and protein). The “C” score is then subtracted from the ‘A’ score to derive the final nutrient profile score. The lower the score the better the nutritional quality of the product. Points are allocated on the basis of the nutritional content in 100 g of a food product or a drink.

Nutrient-rich food (NRF) indices are one method of nutrient profiling based on the concept of nutrient density developed by Fulgoni et al. (Fulgoni et al. J Nutr, 2009. 139(8): p. 1549-54, and has been validated and used in academic research. The NRF9.3 index, calculated per 100 kcal of food item, is based on 9 nutrients to encourage (Protein, dietary, fibre, vitamins A, C, E, Ca, Fe, Mg, K) minus 3 nutrients to limit (saturated fat, total sugar and sodium).

### Validating Scoring System Against Diet Quality (HEI-2010, Energy Density, MAR)

The scoring system was validated against the Healthy Eating Index (HEI-2010), a diet quality score that was developed to measure the conformance to the Dietary Guidelines for Americans 2010 (Guenther et al. J Nutr, 2014. 144(3): p. 399-407). HEI-2010 is made up of 12 components, of which 9 are adequacy components including total fruit, whole fruit, total vegetables, greens and beans, whole grains, dairy, total protein foods, seafood and plant proteins, fatty acids, as well as three moderation components e.g. refined grains, sodium, and empty calories. The total HEI-2010 score has a maximum of 100 points and is based on the sum of these 12 component scores. Data from the National Health and Nutrition Examination Survey (NHANES) 2011-12 were used to calculate HEI. NHANES is a US nationally representative cross sectional survey that collects information on the health and nutrition of the population. Further information on the survey design and methodology of NHANES can be found on the NHANES website (Centers for Disease Control and Prevention 2014). A total of 5075 participants aged 18 years and above were sampled. Dietary data from the first wave of the 24 hour recall interviews were used to calculate HEI-2010 for each participant.

Secondly, Energy density was calculated by dividing the total energy provided by solid foods consumed by each participant by the total edible weight. High ED foods are often linked to poor nutritional quality, therefore a diet high in energy dense products is a less healthy diet.

### Statistical Analysis

At the food level, correlations between the 18 test scores and external nutrient profile scores and subscores (Ofcom, SAIN-LIM and NRF9.3) were tested using Spearman's Rank correlation, to determine the level of agreement between the different profiling systems in ranking the same set of food products. At the diet level, for each participant, energy weighted mean scores were calculated by averaging the food scores of each participant while taking into account the percentage of energy contributed by the products. Similarly, we calculated energy weighted mean Ofcom scores and NRF9.3 scores based on daily food intake. Spearman's Rank correlations were used to compare the agreements between each of the three mean scores and HEI-2010 and ED.

Table 2 displays spearman coefficients correlations between the 18 scores that were tested against energy density of foods, OFCOM score and NRF 9.3.

TABLE 2
Spearman correlations between each score against energy
density, Ofcom and NRF9.3, in the FNDDS table (n = 6960)

| | | | | | | Rank of absolute | | |
|---|---|---|---|---|---|---|---|---|
| | | | | | | correlation with | | |
| Presence of FibreSub | | | Energy | | | Energy | | NRF |
| or Div Ref amount | | | density | Ofcom_score | NRF9.3 | density | Ofcom | 9.3 |
| NO | Sub | Grams | −0.01 | −0.27 | 0.29 | 2 | 1 | 2 |
| | | Serving | −0.21 | −0.33 | 0.26 | 8 | 3 | 1 |
| | | Calories | −0.62 | −0.66 | 0.61 | 14 | 12 | 10 |
| | Div | Grams | 0.05 | −0.28 | 0.30 | 1 | 2 | 4 |
| | | Serving | −0.19 | −0.37 | 0.29 | 6 | 4 | 2 |
| | | Calories | −0.55 | −0.63 | 0.61 | 13 | 9 | 10 |
| Yes | Sub | Grams | −0.19 | −0.61 | 0.61 | 6 | 6 | 10 |
| | | Serving | −0.36 | −0.61 | 0.48 | 10 | 6 | 5 |
| | | Calories | −0.76 | −0.86 | 0.77 | 18 | 17 | 15 |
| | Div | Grams | −0.12 | −0.58 | 0.60 | 3 | 5 | 9 |
| | | Serving | −0.36 | −0.63 | 0.52 | 10 | 9 | 7 |
| | | Calories | −0.68 | −0.81 | 0.77 | 15 | 15 | 15 |
| Yes | Sub | Grams | −0.12 | −0.61 | 0.64 | 3 | 6 | 13 |
| weighted | | Serving | −0.33 | −0.64 | 0.51 | 9 | 11 | 6 |
| by 2 | | Calories | −0.75 | −0.89 | 0.80 | 17 | 18 | 17 |
| | Div | Grams | −0.18 | −0.66 | 0.67 | 5 | 12 | 14 |
| | | Serving | −0.42 | −0.72 | 0.59 | 12 | 14 | 8 |
| | | Calories | −0.69 | −0.85 | 0.80 | 16 | 16 | 17 |

Without information on fibre, calories as reference amount associated with subtraction calculation method performed the strongest correlations with Energy density, OFCOM (respectively −0.62, and −0.66). Correlation with NRF was similar to the division method, still by considering calories as reference amount (spearman correlation coefficient=0.61).

Regardless the reference amount or the method of calculation, integrating fibre in the algorithm significantly improved the correlations coefficients. It was observed that the same reference amount (calories) and method of calculation (subtraction) associated with fibre content weighted by a factor of 2 (Protein+2*fibre−(SFA+sodium+total sugars)) showed the strongest correlations with Energy density, OFCOM and NRF 9.3 (respectively −0.75, −0.89 and 0.80).

The nutrient profile that uses the subtraction method associated with calories as reference amount performs better than others scores tested.

As fibre is not always labelled on products, two of the best algorithms were tested: one with fibre and the next best one without fibre.

Correlation with OFCOM performed better when fibers were included in the score, except for processed fruits and sweets beverages. Adding fibers to the calculation improved especially the correlation with OFCOM for the meat fish and legumes, nuts and seeds categories. Correlation with NRF performed better when fibers were included in the score, except for processed fruits, fats and milk products. Adding fibers to the calculation improved especially the correlation with NRF for the grains and processed vegetables categories. Whatever the score, correlations in fats category were low compared to others.

TABLE 3
Spearman correlations of SCORE_100kcal_
1_sub and SCORE_100kcal_4_sub
with Ofcom and NRF9.3 in each USDA food category

| USDA_ | Ofcom_score with | | NRF9_3 with | |
|---|---|---|---|---|
| catname | kcal_l_sub | kcal_4_sub | kcal_l_sub | kcal_4_sub |
| Eggs | −0.88 | −0.96 | 0.76 | 0.80 |
| (n = 208) | | | | |
| Fats | −0.73 | −0.75 | 0.36 | 0.34 |
| (n = 119) | | | | |
| Grains | −0.89 | −0.92 | 0.49 | 0.64 |
| (n = 1904) | | | | |
| Meat_fish | −0.65 | −0.86 | 0.79 | 0.86 |
| (n = 1989) | | | | |
| Milk | −0.93 | −0.96 | 0.82 | 0.80 |
| products | | | | |
| (n = 435) | | | | |
| legumes_ | −0.77 | −0.90 | 0.70 | 0.75 |
| nuts_seeds | | | | |
| (n = 244) | | | | |
| processed | −0.80 | −0.75 | 0.76 | 0.60 |
| fruits | | | | |
| (n = 332) | | | | |
| processed | −0.65 | −0.75 | 0.70 | 0.80 |
| vegetables | | | | |
| (n = 1301) | | | | |
| sweets_ | −0.95 | −0.94 | 0.50 | 0.53 |
| beverages | | | | |
| (n = 428) | | | | |

NRF, OFCOM and our two selected scores (with and without fibre) were applied to diet of each NHANES respondent in order to provide a weighted by energy average daily food quality score. Table 4 shows the coefficient correlation between those scores and the previously validated HEI. When NRF 9.3 weighted score performed the best against HEI, the selected score including fibers performed better than the OFCOM one.

TABLE 4
Spearman correlations between Ofcom,
NRF9.3, and two selected scores of the invention,
used as indicator for diet against HEI, MAR/2000 kcal and ED.

| | | HEI2010 | Energy density |
|---|---|---|---|
| SCORE_weighted_ofcom | | −0.50634 | 0.64452 |
| | p-value | <.0001 | <.0001 |
| NRF9_3 | | 0.68416 | −0.54108 |
| | p-value | <.0001 | <.0001 |
| SCORE_without fibre | | 0.37896 | −0.61350 |
| | p-value | <.0001 | <.0001 |
| SCORE_with fibre | | 0.51592 | −0.68625 |
| | p-value | <.0001 | <.0001 |
| de_num | | −0.42443 | 1.00000 |
| | p-value | <.0001 | |

As a result, when information on fiber is not labelled, present results suggest that the best equation to estimate the nutritional quality of foods is:

$$( \frac{{PROTEIN}_{100 kcal}}{50} ) - \frac{( \frac{{SFA}_{100 g}}{20} + \frac{T . {SUGARS}_{100 g}}{90} + \frac{{SODIUM}_{100 g}}{2000} )}{3}$$

When fiber content information is available, then the same equation, but adding the fiber content weighted by 2 showed best correlations with previous validated NPs.

$$\frac{( \frac{{PROTEIN}_{100 kcal}}{50} + \frac{2 * {FIBRE}_{100 kcal}}{25} )}{2} - \frac{( \frac{{SFA}_{100 g}}{20} + \frac{T . {SUGARS}_{100 g}}{90} + \frac{{SODIUM}_{100 g}}{2000} )}{3}$$

In summary, it was unexpected that including a fibre component, especially weighting the fibre component in the algorithm significantly improved the correlations coefficients overall. The best performing algorithm including the fibre component showed the strongest correlations with Energy density, OFCOM and NRF 9.3 (respectively −0.75, −0.89 and 0.80). However, if fibre information is not available the product label, the next best algorithm (Protein 100 kcal−(SFA+sodium+total sugars)100 g) could serve the purpose in classifying foods according to their nutritional quality.

1. A method for nutrient scoring food and beverage products wherein the nutrients of the food or beverage product are calculated in a single nutrient score comprising a module for calculation of dietary fibre in a food product or beverage product.

2. A method according to claim 1, wherein the nutrients used to calculate single nutrient score are selected from the group consisting of: fibre, protein, fat, sugar, and sodium.

3. A method according to claim 1, wherein the nutrients used to calculate the single nutrient score are consisting of: fibre, protein, fat, sugar, and sodium.

4. A method according to claim 1, wherein protein and fibre are each calculated per 100 kcal units.

5. A method according to claim 1, wherein fat, sugar and sodium are each calculated according to 100 g units.

6. A method according to claim 1 comprising:
(i) a fibre module configured to calculate the amount of fibre for a product per 100 kcal;
(ii) a protein module configured to calculate the amount of protein for a product per 100 kcal;
(iii) a fat module configured to calculate the amount of fat for a product per 100 g;
(iv) a sugar module configured to calculate the amount of sugar for a product per 100 g;
(v) a sodium module configured to calculate the amount of sodium for a product per 100 g;
(vi) a single product nutrient score module configured to calculate a single nutrient score based on the input of nutrient scores from the modules for fibre, protein, fat, sugar, sodium and
(vii) a user interface display module for causing the at least one display device to display a product nutrient score for said product or plurality of products.

7. A method according to claim 1, wherein the nutrients used to calculate the product nutrient score are calculated according to the formula:
$$\frac{( \frac{{PROTEIN}_{100 kcal}}{50} + \frac{2 * {FIBRE}_{100 kcal}}{25} )}{2} - \frac{( \frac{{SFA}_{100 g}}{20} + \frac{T . {SUGARS}_{100 g}}{90} + \frac{{SODIUM}_{100 g}}{2000} )}{3}$$

8. A method according to claim 1, wherein the nutrients used to calculate the product nutrient score are calculated according to the formula:
$$( \frac{{PROTEIN}_{100 k c a l}}{50} ) - \frac{( \frac{{SFA}_{100 g}}{20} + \frac{T . {SUGARS}_{100 g}}{90} + \frac{{SODIUM}_{100 g}}{2000} )}{3}$$

9. A method of selecting a food or beverage product with the highest product nutrient score by:
(i) comparing the product nutrient score of two or more food or beverage products calculated by the method for nutrient scoring food and beverage products wherein the nutrients of the food or beverage product are calculated in a single nutrient score comprising a module for calculation of dietary fibre in a food product or beverage product
(ii) selecting the food or beverage product with the highest product nutrient score.

10. A computer implemented method for generating food or beverage product recommendations with the highest product nutrient score comprising:
receiving an input of food or beverage product information for nutrients on the label selecting a food or beverage product with the highest product nutrient score by:
(ii) comparing the product nutrient score of two or more food or beverage products calculated by the method for nutrient scoring food and beverage products wherein the nutrients of the food or beverage product are calculated in a single nutrient score comprising a module for calculation of dietary fibre in a food product or beverage product
(iii) selecting the food or beverage product with the highest product nutrient score;
(iv) performing automated, real-time data analysis of the plurality of product nutrient modules and optionally combining it with user preferences
(v) generating product recommendations based on the plurality of product nutrient modules and optionally combining it with user preferences; and
(vi) providing food or beverage product recommendation.
